(19) United States
(12) Patent Application Publication
(10) Pub. No.: US 2008/0146831 A1
(43) Pub. Date: Jun. 19, 2008

(54) Continuous Method for Producing Cyclohexyl(Meth)Acrylate

(75) Inventors: Hugues Vandenmersch, Rayong Province (TH); Mattias Geisendorfer, Neustadt (DE); Horst Hofmann, Eisenberg (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/814,148

(22) PCT Filed: Feb. 10, 2006
(86) PCT No.: PCT/EP06/50834

§ 371 Date: Jul. 17, 2007

(30) Foreign Application Priority Data
Feb. 16, 2005 (DE) 10 2005 006 974.6

(51) Int. Cl. C07C 69/76

(52) U.S. Cl. 560/103

(57) ABSTRACT

A continuous process is proposed for preparing cyclohexyl (meth)acrylate by acid-catalyzed esterification of cyclohexanol with glacial (meth)acrylic acid in the presence of an azeotroping agent for the water of esterification and of a polymerization inhibitor, comprising the following process steps:
esterification of the (meth)acrylic acid (1) with cyclohexanol (2) in the presence of the acidic catalyst (3), of the polymerization inhibitor (4) and of the azeotroping agent for the water of esterification (5) in a reaction zone (A), in which the water of esterification is removed as an azeotrope with the azeotroping agent in a distillation zone attached to the reaction zone (A) to obtain a reaction effluent (process stage A) which
is fed to a neutralization in which the acidic catalyst (3) and unconverted (meth)acrylic acid (1) from the reaction effluent from the esterification are neutralized by means of an alkaline solution to obtain crude cyclohexyl (meth)acrylate (process stage B);
washing of the crude cyclohexyl (meth)acrylate from process stage (B), in which residues of salts are removed from the crude cyclohexyl (meth)acrylate (process stage C), and subsequent distillative workup of the crude cyclohexyl (meth)acrylate (process stages D to G).

The invention relates to a continuous process for preparing cyclohexyl (meth)acrylate by esterifying (meth)acrylic acid with cyclohexanol.

The term (meth)acrylic acid refers in a known manner to acrylic acid and/or methacrylic acid. Analogously, cyclohexyl (meth)acrylate represents cyclohexyl acrylate and/or cyclohexyl methacrylate.

Cyclohexyl (meth)acrylate is a special monomer for the coatings and paints sector. Important applications are, for example, top clearcoats for automobile paint systems, resins for solvent-containing and free coatings, weathering-resistant external paint dispersions and adhesives.

Known processes for the industrial scale preparation of cyclohexyl (meth)acrylate are based predominantly on the transesterification of a (meth)acrylate, in particular methyl (meth)acrylate with cyclohexanol, in the presence of a catalyst. A disadvantage of the transesterification processes is the occurrence of an azeotrope of the (meth)acrylate of the lower alcohol and the lower alcohol, frequently methyl (meth)acrylate/methanol, which has to be worked up in a costly and inconvenient manner or disposed of. In addition, the transesterification process for preparing cyclohexyl (meth)acrylate is unfavorable for kinetic reasons, with correspondingly low space-time yields.

A process for the direct esterification of (meth)acrylic acid with cyclohexanol is described in JP-A 2002-226433. Esterification of (meth)acrylic acid with cyclohexanol in the presence of an acidic catalyst at a molar ratio of cyclohexanol to (meth)acrylic acid of from 1.0:1 to 1.5:1 while azeotropically distilling off the water of esterification with the aid of an azeotroping agent provides a crude ester. In order to remove the catalyst and unconverted (meth)acrylic acid from the crude ester, neutralization is effected with, for example, sodium hydroxide solution. In a first distillation stage, the washed crude ester is freed of high boilers by distillation overhead. In a second distillation stage, the thus obtained distillate is freed of low boilers such as cyclohexanol or azeotroping agent. From the bottom product of the second distillation stage, the target ester, cyclohexyl (meth)acrylate, is distilled overhead in a purifying distillation.

The process described has the particular disadvantage that the repeated distillation of the target ester results in the energy demands being particularly high, and the thermal stress can result in the occurrence of product losses and the formation of secondary components as a result of decomposition of the target ester.

The abovementioned application mentions that the procedure described, with upstream removal of the high boilers and downstream removal of the low boilers, is advantageous for the achievement of a qualitatively high-value pure product compared to processes by which first the low boilers and then the high boilers are removed.

A similar esterification process, but generally for the esterification of (meth)acrylic acid with monohydric alcohols having from 1 to 8 carbon atoms is described by DE-A 196 04 267: in this process too, first the high boilers and only then the low boilers are removed by distillation from the crude product. Accordingly, the target ester is evaporated twice. This document too points out the disadvantage of conventional procedures, by which first the components which are lower-boiling compared to the target ester are removed from the esterification effluent, and that, as a result of the cleavage reactions which, then set in in the bottom of the purifying column in the presence of catalyst and low boilers, it is not possible to obtain low boiler-free and especially (meth)acrylic acid-free pure product.

It was accordingly an object of the invention to provide a process for preparing cyclohexyl (meth)acrylate by direct esterification of (meth)acrylic acid with cyclohexanol, which does not have the disadvantages of the prior art and in particular provides a qualitatively high-value target ester in an energetically favorable process.

The object is achieved by a continuous process for preparing cyclohexyl (meth)acrylate by acid-catalyzed esterification of cyclohexanol with glacial (meth)acrylic acid in the presence of an azeotroping agent for the water of esterification and of a polymerization inhibitor, comprising the following process steps:
esterification of the glacial (meth)acrylic acid (1) with cyclohexanol (2) in the presence of the acidic catalyst (3), of the polymerization inhibitor (4) and of the azeotroping agent for the water of esterification (5) in a reaction zone (A), in which the water of esterification is removed as an azeotrope with the azeotroping agent in a distillation zone attached to the reaction zone (A) to obtain a reaction effluent (process stage A) which
is fed to a neutralization in which the acidic catalyst (3) and unconverted (meth)acrylic acid (1) from the reaction effluent from the esterification are neutralized by means of an alkaline solution to obtain crude cyclohexyl (meth)acrylate (process stage B),
washing of the crude cyclohexyl (meth)acrylate from process stage (B), in which residues of salts are removed from the crude cyclohexyl (meth)acrylate (process stage C),
azeotroping agent distillation of the washed crude cyclohexyl (meth)acrylate from process stage C under reduced pressure and with continuous metered addition of at least one polymerization inhibitor, in the course of which the azeotroping agent, other low boilers and a very small proportion of cyclohexyl (meth)acrylate are drawn off (process stage D),
low boiler removal from the bottom stream from process stage D under reduced pressure and continuous metered addition of at least one polymerization inhibitor to remove the residues of low boilers and a small proportion of the cyclohexyl (meth)acrylate (process stage E),
purifying distillation of the bottom stream from process stage E under reduced pressure and continuous metered addition of at least one polymerization inhibitor to obtain pure cyclohexyl (meth)acrylate and a bottom stream comprising the polymerization inhibitors and also high boilers (process stage F).
residue distillation of the bottom stream from process stage F under reduced pressure and continuous metered addition of at least one polymerization inhibitor, in the course of which residual fractions of cyclohexyl (meth)acrylate are separated from polymerization inhibitors and high boilers (process stage G).

The process according to the invention starts from the reactants cyclohexanol and glacial (meth)acrylic acid.

In the present context, glacial (meth)acrylic acid refers to a (meth)acrylic acid quality which contains at least 98% by weight of (meth)acrylic acid or else with at least 99.5% by weight of (meth)acrylic acid, and additionally not more than 0.2% by weight of water and also in each case not more than 0.03% by weight of acetic acid, propionic acid and isobutyric acid.

Preference is given to using a cyclohexanol quality comprising at least 98.5% by weight of cyclohexanol, with not more than 0.25% by weight of cyclohexanone, not more than 0.3% by weight of cyclohexyl acetate, not more than 5 mg of phenol based on 1 kg of cyclohexanol, and not more than 0.1% by weight of water.

### Esterification of the Glacial (meth)acrylic Acid with Cyclohexanol (Process Stage A)

The glacial (meth)acrylic acid and cyclohexanol reactants are fed continuously to a suitable reaction zone which may either be an individual reactor or a battery of two or more successive reaction regions, in which case the discharge stream of one reaction region forms the feed stream of the next reaction region. In the embodiment with a plurality of reaction regions, preference is given to feeding the ascending vapors from all reaction regions to a single distillation apparatus whose liquid effluent is recycled only into the first reaction region.

The reaction units may be separate reactors or else different regions in one reactor.

The reactors used may be stirred tanks or stills which are equipped with heating spirals or jackets and have external natural-circulation or forced-circulation evaporators.

A distillation column for the removal of the water of esterification is attached to the first reactor.

Advantageously, the ascending vapors from all reaction regions may be fed to a single distillation column whose effluent is fed only into the first reaction region. However, it is also possible to equip all reaction vessels with in each case a dedicated attached distillation column.

A distillation column may be a column having random packing, a column having structured packing or a tray column, preferably having from 1 to 15 theoretical plates.

Glacial (meth)acrylic acid and cyclohexanol are used preferably in a molar ratio in the range from 0.9 to 2.0, in particular in the range from 1.05 to 1.15.

Suitable acidic esterification catalysts are in particular sulfuric acid, para-toluenesulfonic acid or other organic sulfonic acids, particularly methanesulfonic acid, benzenesulfonic acid or dodecylbenzenesulfonic acid.

The acidic esterification catalyst is fed preferably in a concentration of from 1 to 5% by weight based on the glacial (meth)acrylic acid used.

The azeotroping agent used for the water of esterification is preferably a substance or a mixture of substances selected from the following list: cyclohexane, cyclohexene, methylcyclohexane, benzene, toluene or hexanes.

The polymerization inhibitor used is advantageously a substance or a mixture of substances in a concentration in the range from 100 to 5000 ppm based on the effluent from the reaction zone, selected from the following list: phenothiazine, 4-nitrosophenol, 4-hydroxy-2,3,6,6-tetramethylpiperidine N-oxyl, hydroquinone or hydroquinone monomethyl ether.

Advantageously, oxygen may be used additionally as a polymerization inhibitor.

The reaction in process stage A is effected either under standard pressure or under reduced pressure, at a temperature between 70 and 160° C.,

To the reaction zone one or more distillation columns are attached. The distillate obtained therein is condensed and separated in a phase separator into an organic and an aqueous phase. The aqueous phase is either added to the wastewater which is in need of treatment or preferably sent to the washing in process stage C.

The organic phase which comprises the azeotroping agent is recycled as reflux to the distillation column(s) and, if appropriate, also directly into the reaction zone.

The esterification effluent from the reaction zone comprises the target ester, unconverted reactants, catalyst, polymerization inhibitor(s) and by-products.

Possible by-products are in particular cyclohexanone, cyclohexyl acetate and cyclohexyl propionate.

The esterification effluent is preferably cooled in a heat exchanger to a temperature of from 20 to 40° C. and subsequently sent to the neutralization (process stage B).

### Neutralization (Process Stage B)

In process stage B, the esterification effluent is freed from the catalyst and from unconverted (meth)acrylic acid with the aid of an alkaline solution, in particular sodium hydroxide solution, potassium hydroxide solution or sodium carbonate.

The neutralization is preferably carried out in mixer-settlers. The aqueous phase is sent to the wastewater in need of treatment, while the organic phase is sent to the next process stage, the washing (process stage C).

### Washing (Process Stage C)

In this stage, the organic phase from the neutralization is freed of salts with the aid of a washing solution, in particular water, which may advantageously be water from the phase separator of the reaction zone, i.e. process water. The aqueous phase is preferably sent to the wastewater in need of treatment, in apparatus terms, process stage C, like process stage B too, is carried out in mixer-settlers. Possible settlers for the neutralization (process stage B) and also for the washing (process stage C) are, for example, decanters or extraction columns.

The washed crude ester obtained in the washing (neutral ester) is worked up in a series of distillation stages:

### Azeotroping Agent Distillation (Process Stage D)

In the azeotroping agent distillation, the azeotroping agent used to remove the water of esterification in the reaction zone is distilled overhead and preferably recycled for the most part into the esterification (process stage A). A small portion of the distillate is discharged in order to prevent an accumulation of impurities.

Possible apparatus for the azeotroping agent distillation includes, for example, columns having random packing, columns having structured packing or tray columns having preferably from 1 to 5 theoretical plates. The azeotroping agent distillation is preferably operated at a top pressure between 60 and 150 mbar, more preferably at a top pressure between 70 and 100 mbar.

### Low Boiler Removal (Process Stage E)

In the low boiler removal, remaining low boilers are distilled overhead out of the bottom product of the azeotroping agent distillation (process stage D) and preferably recycled into the neutralization (process stage B) or into the esterification (process stage A).

Possible apparatus for the low boiler removal includes, for example, columns having random packing, columns having structured packing or tray columns, preferably having from 1 to 15 theoretical plates. The low boiler removal is preferably carried out at a top pressure of from 5 to 80 mbar. in particular at a top pressure between 5 and 50 mbar.

It is possible to carry out the azeotroping agent distillation (process stage D) and the low boiler removal (process stage E) in a combined distillation unit.

### Purifying Distillation (Process Stage F)

In the purifying distillation, the pure cyclohexyl (meth)acrylate is obtained in vapor form from the bottom product of the low boiler removal (process stage E) and stabilized with a storage stabilizer. An example of a possible storage stabilizer is hydroquinone monomethyl ether. Possible distillation apparatus for the purifying distillation includes, for example, columns having random packing, columns having structured packing or tray columns, in particular having from 1 to 15 theoretical plates, or else a thin-film evaporator. The purifying distillation is operated preferably at a top pressure in the range from 1 to 20 mbar, more preferably at a top pressure in the range from 1 to 5 mbar.

### Residue Distillation (Process Stage G)

In the residue distillation, fractions of the cyclohexyl (meth)acrylate target ester which are still present are distilled overhead out of the bottom product of the purifying distillation and recycled into the purifying distillation. Possible apparatus includes columns having random packing, columns having structured packing or tray columns, in particular having from 1 to 15 theoretical plates, or else thin-film evaporators.

The purifying distillation is carried out preferably at a top pressure in the range from 1 to 20 mbar, more preferably at a top pressure in the range from 1 to 5 mbar.

From the purifying distillation, pure cyclohexyl (meth)acrylate is obtained, pure cyclohexyl (meth)acrylate referring in the present context to a pure cyclohexyl (meth)acrylate quality having at least 98% by weight of cyclohexyl (meth)acrylate, not more than 1000 ppm of water, not more than 100 ppm of (meth)acrylic acid, a color number of <10 APHA and 50 +/−5 ppm of hydroquinone monomethyl ether.

The distillation apparatuses used in the individual distillation stages D to G each comprise an evaporator and a condensation unit. The evaporators may be natural-circulation or forced-circulation evaporators, falling-film evaporators or thin-film evaporators. Possible condensation units include, for example, tube bundle heat exchangers, plate heat exchangers or direct condensers (quench apparatuses).

In all process stages, polymerization inhibitors are added against undesired polymerization. Possible polymerization inhibitors include, for example, phenothiazine, para-nitrosophenol, copper(I) chloride, copper(II) chloride or hydroquinone monomethyl ether or else mixtures thereof. The polymerization inhibitor(s) is/are added as a solution.

In this context, possible solvents for the polymerization inhibitor(s) include pure cyclohexyl (meth)acrylate or else the corresponding crude esters or washed crude esters (neutral esters). The concentration of polymerization inhibitor in the solution is between 0.1 and 2.0% by weight. This solution is preferably fed directly to the particular distillation columns, preferably via the reflux line and/or the condensers at the top of the column.

The vacuum in the individual distillation columns may be generated by steam ejectors or liquid-ring pumps which are operated, for example, with water.

The residues from the azeotroping agent distillation and from the residue distillation may, for example, be utilized thermally in a suitable incineration plant. The offgases coming from the plant may be disposed of, for example, in a flare.

The invention is illustrated in detail below with reference to a drawing and to a working example.

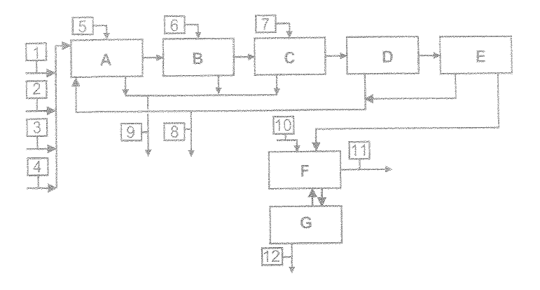

FIG. 1 shows the schematic representation of a preferred plant for carrying out the process according to the invention.

In the single FIGURE, process stages A to G are indicated with connecting arrows which symbolize streams. The large arrows symbolize the main stream to the target ester, the pure cyclohexyl (meth)acrylate.

A stream 1 comprising glacial (meth)acrylic acid, a stream 2 comprising cyclohexanol, a stream 3 comprising acidic catalyst, a stream 4 comprising polymerization inhibitor and a stream 5 comprising azeotroping agent are fed into process stage A, the esterification.

The main stream from process stage A is passed into process stage B, the neutralization, in which a neutralization is effected with addition of an alkali solution, stream 6. The main stream from the neutralization is passed into process stage C, the washing, in which a washed crude ester is obtained with supply of a wash solution, stream 7, and is subsequently passed into process stage D, the azeotroping agent distillation. From the azeotroping agent distillation, a stream comprising azeotroping agent can be recycled into process stage A, the esterification. The main stream from the azeotroping agent distillation is passed into process stage E, the low boiler removal. From this removal, a substream can be recycled into the neutralization or into the washing. The main stream from the low boiler removal, process stage E, is passed into the purifying distillation, process stage F. Storage stabilizer, stream 10, is fed to process stage F, and the target ester, pure cyclohexyl (meth)acrylate, stream 11, is drawn off in vapor form. The bottom stream from the purifying distillation is worked up further in a residue distillation, process stage G, from which a high boiler residue, stream 12, is discharged.

### WORKING EXAMPLE

In a process for continuously preparing cyclohexyl (meth)acrylate, 163.3 g/h of methacrylic acid having a content of phenothiazine as a process stabilizer of 0.1% by weight, based on the weight of methacrylic acid, 5.4 g/h of a 65% aqueous solution of para-toluenesulfonic acid and 181.3 g/h of cyclohexanol were metered continuously to the first reactor of a three-stage reactor battery with natural-circulation evaporators. The reactants corresponded in each case to the quality specified below, the components being specified in % by weight:

methacrylic acid with

99.54% methacrylic acid.

0.15% acrylic acid,

0.03% propionic acid,

0.03% acetic acid.

9.2% water,

0.03% isobutyric acid and

0.02% hydroquinone monomethyl ether, and

cyclohexanol with

99.35% cyclohexanol,

0.1% water,

0.25% cyclohexanone and

0.30% cyclohexyl acetate.

Additionally fed continuously to the first reactor of the battery was a circulation return stream from the top of the azeotroping agent distillation (process stage D) of 127.5 g/h and of the following composition:

6.5% by weight of cyclohexanol,

20.0% by weight of cyclohexyl methacrylate,

3.2% by weight of cyclohexanone,

1.3% by weight of cyclohexyl acetate and

68.6% by weight of cyclohexane.

843.7 g/h of cyclohexane were introduced as reflux to the distillation column which was attached to the first reactor of the battery as an azeotroping agent for removing the water of esterification. Furthermore, an additional 260.2 g/h of cyclohexane were fed to each reactor of the battery to the natural-circulation evaporators of the individual reactors of the battery.

The molar methacrylic acid/cyclohexanol ratio in the reactor feed was 1.025:1.

At a residence time of 24 hours, a reaction temperature of 120° C. in the reactors of the battery was established.

From the third reactor of the battery, a reaction effluent of 46i with the composition listed below was drawn off: 2% by weight of methacrylic acid, 2% by weight of cyclohexanol, 71.6% by weight of cyclohexyl methacrylate, 1.0% by weight of cyclohexanone, 0.5% by weight of cyclohexyl acetate and 20.0% by weight of cyclohexane.

The reactor effluent was cooled to a temperature of 30° C. and neutralized with a 10% aqueous sodium carbonate solution in process stage B. The molar ratio of sodium carbonate to methacrylic acid+para-toluenesulfonic acid was 2:1.

The neutral ester obtained in process stage B was freed of residual salts and washed with 804 g/h of water in a mixer-settler apparatus in process stage C. The washed neutral ester was fed to process stage D, the azeotroping agent distillation. The cyclohexane-enriched distillate was fed into the still of the first esterification reactor down to a small substream of 10.0 g/h which was discharged. The azeotroping agent distillation was carried out at a top pressure of 80 mbar and a bottom temperature of 128° C. in a rectification column equipped with size 35 Pall rings.

The bottom effluent from the azeotroping agent distillation was distilled at a top pressure of 50 mbar and a bottom temperature of 113° C. in a distillation column equipped with size 25 Pall rings in process stage E. the low boiler removal.

The distillate enriched in low boilers, especially cyclohexanol, cyclohexanone and cyclohexyl acetate, was recycled into process stage B, the neutralization. Since the low boilers were removed virtually fully in process stage E, a crude ester was obtained in a mass flow rate of 338.0 g/h in the bottom effluent of the low boiler removal and already comprised 96.9% by weight of cyclohexyl methacrylate and also stabilizer.

In process stage F, the purifying distillation, in a first, relatively large thin-film evaporator, at an evaporative concentration ratio (ratio of vapor to feed stream) of approx. 75%, a top stream of 280.0 g/h of pure cyclohexyl methacrylate having the following composition was obtained:

99.5% by weight of cyclohexyl methacrylate, less than 1000 ppm of cyclohexanol, less than 100 ppm of methacrylic acid, less than 500 ppm of water and a color number of <10 APHA. The pure cyclohexyl methacrylate was stabilized for storage by addition of hydroquinone monomethyl ether in a concentration of 50 ppm.

In a second, smaller thin-film evaporator, which was operated at an evaporative concentration ratio of approx. 80%, a high boiler residue of approx. 18.75 g/h was obtained in the residue distillation (process stage G). The distillate from the residue distillation was recycled into the purifying distillation.

The reactant yields based on the overall process were 86% for methacrylic acid and 92% for cyclohexanol.

1. A continuous process for preparing cyclohexyl (meth)acrylate by acid-catalyzed esterification of cyclohexanol with glacial, (meth)acrylic acid in the presence of an azeotroping agent for the water of esterification and of a polymerization inhibitor, comprising the following process steps:
esterification of the glacial (meth)acrylic acid with cyclohexanol in the presence of the acidic catalyst, of the polymerization inhibitor and of the azeotroping agent for the water of esterification in a reaction zone (A), in which the water of esterification is removed as an azeotrope with the azeotroping agent in a distillation zone attached to the reaction zone (A) to obtain a reaction effluent (process stage A) which
is fed to a neutralization in which the acidic catalyst and unconverted (meth)acrylic acid from the reaction effluent from the esterification are neutralized by means of an alkaline solution to obtain crude cyclohexyl (meth)acrylate (process stage B);
washing of the crude cyclohexyl (meth)acrylate from process stage B, in which residues of salts are removed from the crude cyclohexyl (meth)acrylate (process stage C);
azeotroping agent distillation of the washed crude cyclohexyl (meth)acrylate from process stage C under reduced pressure and with continuous metered addition of at least one polymerization inhibitor, in the course of which the azeotroping agent, other low boilers and a very small proportion of cyclohexyl (meth)acrylate are drawn off (process stage D);
low boiler removal from the bottom stream from process stage D under reduced pressure and continuous metered addition of at least one polymerization inhibitor to remove the residues of low boilers and a small proportion of the cyclohexyl (meth)acrylate (process stage E);
purifying distillation of the bottom stream from process stage E under reduced pressure and continuous metered addition of at least one polymerization inhibitor to obtain pure cyclohexyl (meth)acrylate and a bottom stream comprising the polymerization inhibitors and also high boilers (process stage F);
residue distillation of the bottom stream from process stage F under reduced pressure and continuous metered addition of at least one polymerization inhibitor, in the course of which residual fractions of cyclohexyl (meth)acrylate are separated from polymerization inhibitors and high boilers (process stage G).

2. The process according to claim 1, wherein the top stream from process stage D is partly discharged and otherwise recycled into reaction zone (A), and/or the top stream from process stage E is recycled into process stage B or A.

3. The process according to claim 1, wherein reaction zone (A) is formed from two or more reaction regions connected in series and the discharge stream of one reaction region forms the feed of the downstream reaction region.

4. The process according to claim 3, wherein the vapors rising out of the two or more reaction regions connected in series are fed to a single distillation column whose liquid effluent is recycled into the first reaction region.

5. The process according to claim 1, wherein a molar ratio of glacial (meth)acrylic acid to cyclohexanol in the feed to reaction zone (A) between 0.9 and 2.0.

6. The process according to claim 1, wherein the acidic esterification catalyst is sulfuric acid, para-toluenesulfonic acid or another organic sulfonic acid, in particular methanoic acid.

7. The process according to claim 1, wherein the catalyst is in a proportion of from 1 to 5% by weight based on the weight of glacial (meth)acrylic acid used.

8. The process according to claim 1, wherein the azeotroping agent is one substance or a mixture of substances selected from the following list: cyclohexane, cyclohexene, methylcyclohexane, benzene, toluene or hexanes.

9. The process according to claim 1, wherein the polymerization inhibitor is one substance or a mixture of substances selected from the following list: phenothiazine, 4-nitrosophenol, 4-hydroxy-2,3,6,6-tetramethylpiperidine N-oxyl, hydroquinone or hydroquinone monomethyl ether, and the amount of the polymerization inhibitor is in the range from 100 to 5000 ppm based on the effluent from reaction zone (A).

10. The process according to claim 9, wherein oxygen is used additionally as the polymerization inhibitor.

11. The process according to claim 1, wherein process stage A is carried out at standard pressure and a temperature in the range from 70 to 140° C.

12. The process according claim 3, wherein the pressure and the temperature are the same in all reaction regions.

13. The process according to claim 1, wherein the residence time in reaction zone (A) is from 5 to 30 hours.

14. The process according to claim 3, wherein the two or more reaction regions are each formed from one reactor having one circulation evaporator.

15. The process according to claim 1, wherein the azeotroping agent for the water of esterification is supplied via the distillation column attached to reaction zone (A).

16. The process according to claim 15, wherein the azeotroping agent is fed additionally to reaction zone (A) or to each of the two or more reaction regions.

17. The process according to claim 1, wherein the aqueous phase obtained at the top of the distillation column attached to reaction zone (A) is discharged substantially fully.

18. The process according to claim 1, wherein the alkaline solution used in process stage B is an aqueous sodium carbonate, sodium hydroxide or potassium hydroxide solution.

19. The process according to claim 1, wherein process stages B and C are carried out in mixer-settlers.

20. The process according to claim 1, wherein process stage D is carried out at a top pressure in the range from 60 to 150 mbar.

21. The process according to claim 1, wherein process stage D is carried out in a column having random packing.

22. The process according to claim 1, wherein process stage E is carried out at a top pressure in the range from 5 to 80 mbar.

23. The process according to claim 1, wherein process stage E is carried out in a column having random packing.

24. The process according to claim 1, wherein process stage F is carried out at a top pressure in the range from 1 to 20 mbar.

25. The process according to claim 1, wherein process stage F is carried out in a thin-film evaporator.

26. The process according to claim 1, wherein process stage G is carried out at a top pressure in the range from 1 to 20 mbar.

27. The process according to claim 1, wherein process stage G is carried out in a thin-film evaporator.
